(19) United States
(12) Patent Application Publication
(10) Pub. No.: US 2023/0347047 A1
(43) Pub. Date: Nov. 2, 2023

(54) CONTROL SYSTEM FOR INFUSION PUMP

(71) Applicant: Pascal LIZOTTE, Montreal (CA)

(72) Inventor: Pascal LIZOTTE, Montreal (CA)

(21) Appl. No.: 17/731,631

(22) Filed: Apr. 28, 2022

(51) Int. Cl. A61M 5/168; A61M 5/14; A61M 5/148

(52) U.S. Cl. CPC A61M 5/16877; A61M 5/1415; A61M 5/148; A61M 2005/1405

(57) ABSTRACT

A control system for an infusion pump includes a converter electrically coupled to the infusion pump and an actuator configured for receiving a mechanical force. The actuator is further configured for selectively transmitting the mechanical force to the converter indicative of a patient's request for a bolus infusion. The converter is configured for converting the mechanical force received from the actuator to an electrical signal transmission to the infusion pump.

### TECHNICAL FIELD

This disclosure generally relate to the field of infusion pumps, and more particularly to the field of actuators for infusion pumps.

### BACKGROUND

Infusion pumps are medical devices, often found in clinical settings such as hospitals and nursing homes, that deliver fluids in controlled amounts, such as nutrients and medications, into a patient's body. One such example of an infusion pump is a patient-actuated analgesia pump, which allows a patient, for instance post-surgery, to control the pain medication delivery themselves via a hand-held actuator. By allowing the patient to self-administer a dose at a desired time, an optimal level of pain relief may be enabled, while avoiding over or under-dosage.

However, patients requiring pain medication may have limited mobility, for instance due to a disability, or as a result of an injury. This limited mobility may prevent them from having the necessary physical faculties or dexterity to manipulate and activate the hand-held actuator of the infusion pump, for instance via the relatively small contact surface at a distal end of the actuator or on the bedside infusion pump, thus preventing them from administering their dosage. A third party individual may then be required to administer the patient's dosage, such as a family member, a friend, or a medical professional. However, these third party individuals may not be present when a dosage is needed. Therefore, improvements are needed.

### SUMMARY

In accordance with one aspect, there is provided a control system for an infusion pump, comprising: a converter electrically coupled to the infusion pump; and an actuator configured for receiving a mechanical force, and for selectively transmitting the mechanical force to the converter indicative of a patient's request for a bolus infusion; wherein the converter is configured for converting the mechanical force received from the actuator to an electrical signal transmission to the infusion pump.

In accordance with another aspect, there is provided a method for controlling the operation of an infusion pump, comprising: receiving, at a converter, a mechanical force transmitted by an actuator, the mechanical force indicative of a patient request for an bolus infusion; converting, at the converter, the mechanical force to an electrical signal transmission; and transmitting the electrical signal from the converter to the infusion pump.

Many further features and combinations thereof concerning the present improvements will appear to those skilled in the art following a reading of the instant disclosure.

### BRIEF DESCRIPTION OF THE DRAWINGS

FIG. 1 is a block diagram of a control system for an infusion pump according to an embodiment;

FIG. 2 is a top view of the control system of FIG. 1;

FIGS. 3A-3B are respective perspective and top views of a converter for the control system of FIG. 1; and

FIG. 4 is a flowchart showing an exemplary method for controlling the operation of an infusion pump according to an embodiment.

### DETAILED DESCRIPTION

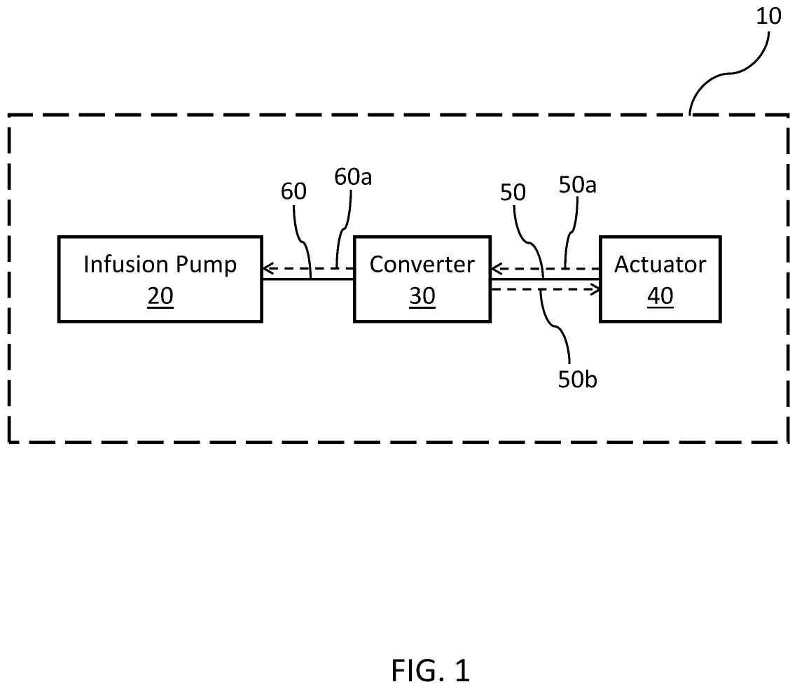

Referring to FIG. 1, there is shown a control system 10 for an infusion pump 20, according to an exemplary embodiment of the present disclosure. In the shown embodiment, the infusion pump 20 is a patient-controlled analgesia pump configured for delivering a pre-determined dosage or bolus of medication to a patient. Such medication may be pain medication, for instance to be delivered to a patient after the patient has undergone surgery, a treatment, or as part of a therapy, as examples among others. Other types of infusion pumps and circumstances for their use may be contemplated. The control system 10 includes a converter 30 electrically coupled to the infusion pump 20, and an actuator 40 engageable by a patient and configured for selectively transmitting a mechanical force, such as a fluid-based signal, to the converter 30 indicative of the patient's request for a bolus infusion. As will be discussed in further detail below, actuator 40 may be referred to as an ergonomic actuator, as it is engageable by a patient with limited mobility.

While FIG. 1 shows the infusion pump 20 to be included in the control system 10, it is understood that the control system 10 may alternatively exclude the infusion pump 20 and include different combinations of a converter 30 or like interface, an actuator 40, a fluid line 50, and/or an electrical line 60 configured to interact with an existing infusion pump 20, for instance an infusion pump 20 currently in use at a medical facility and operatively coupled to the control system 10. In other cases, a control system 10 including a converter 30 and actuator 40 may be adapted for other implementations requiring self-monitoring for a patient with disabilities and/or motor difficulties. As will be discussed in further detail below, the converter 30 is configured for converting the fluid-based signal 50a (or other mechanical force, as will be discussed in further detail below) received from the actuator 40, illustratively via fluid line 50, to an electrical signal 60a to be transmitted to the infusion pump 20, illustratively via electrical line 60.

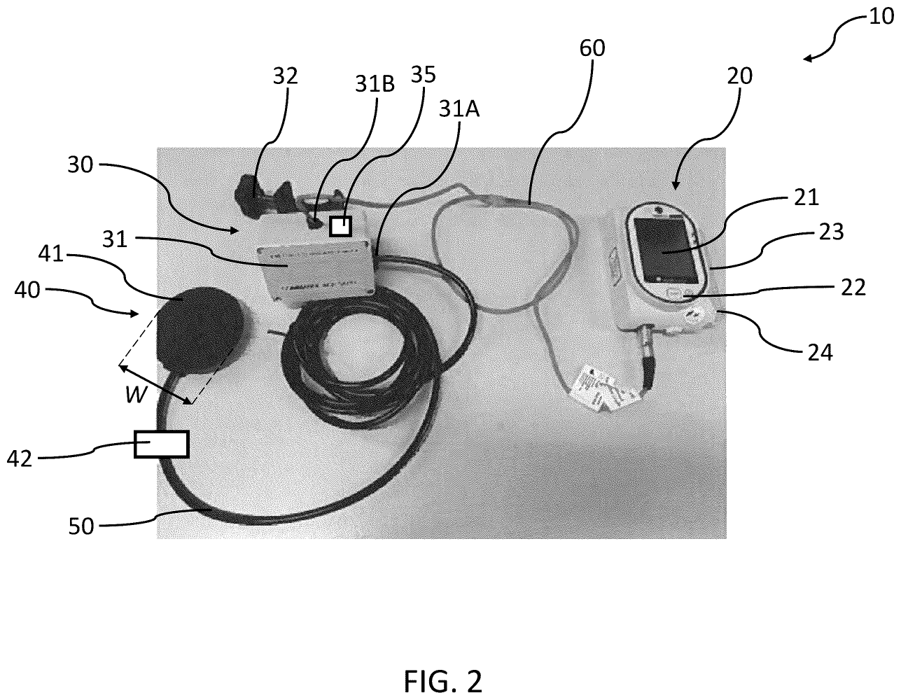

Referring to FIG. 2, the infusion pump 20 is illustratively shown to be a patient-controlled analgesia pump. The pump 20 may be configured to deliver various pain (or other) medications to a patient in pre-determined dosages when the actuator 40 is actuated. The pump 20 may thus include hardware and software, for instance memory, a printed circuit board, a power source such a battery, and a user interface, to control the dosage to be delivered to a patient. The user interface may include a screen 21 and/or one or more buttons 22 on an exterior surface of the pump 20. In some embodiments, the user interface may allow a user, for instance a medical professional, to adjust the volume of each dose, the type of medication provided to the patient, the increments of time between permissible doses, the maximum number of dosages a patient may receive in a given period of time, as well as other functions that may prevent a patient overdose. Other functions for the pump 20 may be contemplated. The user interface may additionally permit a user to temporarily restrict access to further doses. In some cases, the screen 21 may be responsive to touch input. The pump 20 may include an inlet 23 through which medication is received at the pump 20 and a pumping mechanism (not shown) disposed inside the pump 20 for delivering the medication. An outlet 24 may be fluidly coupled to a tube (not shown) to selectively deliver a dosage of medication to a patient when required, for instance via intravenous delivery.

In the shown embodiment, the converter 30 includes an enclosure or box 31 containing the components required to convert the fluid-based signal 50a received from the actuator 40 to an electrical signal 60a to be transmitted to the infusion pump 20. The enclosure 31 may be substantially waterproof or otherwise sealed to prevent fluids from accidentally entering its internal cavity. In such cases, the enclosure may include a door or removable panel to provide access to its internal cavity while sealing it when closed. The shown enclosure 31 may include an inlet 31A for the fluid line 50 and an outlet 31B for the electrical line 60. The enclosure 31 may further include a mount 32 for securely positioning the enclosure 31 in the vicinity of the patient and the infusion pump 20. Other mounting means for enclosure 31, for instance fasteners or adhesives, may be contemplated. In an embodiment, the enclosure 31 may be mounted to a solute pole (not shown) for the infusion pump 20. Other mounting locations for the enclosure, for instance on a recovery bed of the patient, may be contemplated.

Still referring to FIG. 2, in the shown embodiment, the actuator 40 includes a circular (or other shape) user interface 41 fluidly coupled to a first end of the fluid line 50. User interface 41 is configured for transmitting a fluid-based signal 50a to the converter 30 via fluid line 50, the fluid-based signal 50a being for example a pneumatic or hydraulic force. In the shown embodiment, the user interface 41 is a bladder 41 and the fluid line 50 is a pneumatic tube. As such, a patient may press the bladder 41 to transmit a pneumatic signal via pneumatic tube 50 to the converter 30. Other fluid-based signals may be contemplated. For instance, the bladder 41 and fluid line 50 may be filled with a liquid, such as water or oil, rather than air, and the fluid-based signal 50a may be a hydraulic signal. In other cases, the fluid-based signal 50a may be replaced with other mechanical-based signals, for instance a signal transmitted by components such as a cable and housing set, spring(s), levers and/or elastics that are triggered by actuation of the bladder 41. Such fluid or mechanical-based signals, i.e., non-electrical signals, may avoid any electrical interference with the pump 20. Moreover, because of the use of mechanical-based signals, the converter 30 may be said to be passive, in that it does not include a power source. While the electrical line 60 entering the converter 30 may be powered, it may be low voltage and it may be configured to avoid electrical hazards by complying with applicable standards.

The positioning and dimensions of the actuator 40 are configured for allowing patients with limited mobility to easily actuate the actuator 40 to administer a dose of their medication themselves. By limited mobility, it is understood that the patient may be unable to manipulate a typical hand or finger controlled button or actuator for a patient-controlled analgesia pump due to, for instance, a physical disability, or an injury. Other considerations may factor into a patient's limited mobility as well, for instance any temporary limitations following surgery. In some cases, a patient may simply lack the dexterity required to manipulate the small button typically found on a hand-held actuator associated with an infusion pump. As such, the actuator 40, and more particularly the user interface 41, may be positioned adjacent or within reach of one of the patient's mobile bodily parts, such as their head, their elbow, their forearm, their hand, their knee, or their foot. Other bodily parts may be contemplated.

The user interface 41 may be mounted to a recovery bed (not shown) of the patient, for instance on an inner guard rail adjacent to one of the above-mentioned bodily parts. A recovery bed may refer to, for instance, a hospital bed for a patient post-surgery. Similarly, a recovery bed may refer to a medical-type bed in a rehabilitation center, nursing home or a private residence. Other recovery bed types may be contemplated, or other patient supports may be provided with the actuator 40, such as chairs, wheelchairs, stretchers, etc. Alternatively, the user interface 41 may be mounted to a bedpost or a portion of a bedframe. The positioning of the user interface 41 may depend on the specific circumstances of each patient and the specific bodily parts they are most adept at using to actuate the actuator 40.

In some embodiments, fixing means 42 may be provided adjacent the user interface 41 or proximate the user interface 41 along the fluid line 50 (as schematically shown in FIG. 2) to mount the user interface 41 to, for instance, a patient recovery bed, chair, wheelchair, stretcher, etc. Such fixing means 42 may include, for instance, a clamp, a clip, a tie, or a hook-and-loop fastener. In other cases, the fixing means 42 may be integrated with the user interface 41. For instance, the user interface 41 may include fixing or attaching means such as an adhesive strip on a rear side of the user interface 41 (i.e., opposite the surface or side of the user interface 41 activated by the patient). Other fixing means may be contemplated. In the illustrated embodiment of FIG. 2, the use of a bladder as user interface 41 may allow its positioning in close proximity to the client, such that body weight may be used to depress the bladder. The bladder 41 may be without the fixing means 42, or the fixing means 42 may be present to attach the fluid line 50 to a surrounding structure.

The shape and dimensions of the user interface 41 may be selected to ensure that the patient with limited mobility may successfully actuate, i.e., press, the user interface 41. The user interface 41 (and thus the actuator 40) may thus be referred to as ergonomic, as the contact surface which, when engaged, actuates the user interface 41 is large enough to be engaged by one or more of the above-mentioned bodily parts. This is in contrast to typical buttons for infusion pumps, which typically include small buttons at a distal end of an actuator that are difficult to manipulate for patients with limited mobility and can typically be only manipulated with one's fingers.

In the non-limiting example shown in FIG. 2, the user interface 41 is circularly-shaped. Other shapes for the user interface 41 may be contemplated. A width W of the user interface 41 (or diameter in the case of a circular bladder) may be selected to ensure a patient may successfully activate it with a bodily part other than their fingers, for instance their knee or their elbow. For instance, in a non-limiting example, the user interface 41 may have a width W of approximately 9 centimeters or greater. In addition, the width W of the user interface may be selected to ensure that it is not pressed or activated inadvertently. For instance, in a non-limiting example, the user interface 41 may have a width W of less than 20 centimeters. Other ranges for the width W may be contemplated as well. A depth or thickness of the user interface 41 may vary, for instance based on the type of fluid utilized for the fluid-based signal 50a being transmitted to the converter 30. For instance, in a non-limiting example, the shown pneumatic user interface 41 has a depth of approximately 1.5 centimeters, although other depths may be contemplated. The user interface 41 may have a width W that exceeds its depth, ensuring that the user interface 41 has a large surface area for easy engagement without overly protruding into the patient's bed area. When the user interface 41 is a bladder, its size may be determined on its fluid content, such as at least 50 ml of fluid.

Other considerations to avoid inadvertent activation of the user interface 41, while ensuring the user interface 41 is easy to activate by a patient, may be contemplated. For instance, to activate the user interface 41, a combination of a low activation force (thereby facilitating the task of the patient) with a long stroke of movement (i.e., a long press of the interface) and/or a large surface of force may be required. In other words, the user interface 41 may require a relatively substantial displacement of fluid without substantial force to ensure that a patient will only activate the user interface 41 when such action is actually intended. This may prevent inadvertent contacts with the user interface 41 being registered as activations, as such contacts would not create sufficient displacement of fluid in the user interface 41. The depth of the user interface 41 or size of the bladder 41 may be increased to allow for this long stroke of movement or large surface of pressure, and the volume of fluid contained in the user interface 41 and/or the material of the user interface 41 may be selected to provide a low activation force for activating the user interface 41.

The user interface 41 may be configured to activate, i.e. transmit the fluid-based signal 50a to the converter 30, regardless of how the patient presses it (ex: force, speed, intensity or duration of application). The user interface 41 may be described as having a single function, i.e., bolus administration, once the user interface 41 is pressed, provided that the infusion pump 20 allows the administration according to the patient's prescription profile. This may ease the task of activating the user interface 41 for the patient given their limited mobility. In addition, in cases where the user interface 41 is configured for transmitting a pneumatic signal to the converter 30, the user interface 41 may be soft and easily pressed, easing the task of the patient as well and requiring little pressure to transmit the signal. Alternatively, the rigidity of the user interface 41 may be increased, for instance by using a stronger material and/or by replacing the air inside the user interface 41 with a liquid, to reduce the chances of accidental activation of the user interface 41. In various cases, different user interfaces 41 with different dimensions and/or rigidities may be produced based on the bodily part of the patient intended to be used with the user interface 41.

Referring additionally to FIG. 1, an opposing mechanical force 50b may be received or applied against the user interface 41 subsequently to the user interface's 41 activation by a patient. This opposition force 50b may prepare the user interface 41 for future activation(s) by the patient, for instance by allowing the user interface 41 to regain its original (i.e., non-pressed) form. The opposition force 50b may take different forms, and may originate from the converter 30 and/or from the actuator 40 itself. In some cases, the opposing force 50b may be in the same form as the mechanical force transmitted by the actuator 40 to the converter 30 (e.g, pneumatic). The alternative may be true as well, i.e., the opposing force 50b may take a different form than the transmitted mechanical force to the converter 30. In an embodiment, the user interface 41 may be spring-loaded, urging the outer surface of the user interface 41 back to its original form after the patient has completed an activation action. If the user interface 41 is a bladder, it may have a spring back effect to return to its native shape. As another example, the user interface 41 may include an internal spring configured for biasing the surface of the user interface 41 after the user interface 41 is released by the patient. Additionally or alternatively, the material forming the surface of the user interface 41 may be configured to provide the necessary spring-back effect. Other opposition forces 50b within the actuator 40 may be contemplated. Additionally or alternatively, and as depicted in FIG. 1, the opposition force may originate from the converter 30. For instance, in the case of a fluid-based signal 50a, the converter 30 may include a bladder, diaphragm, or other like device for returning the fluid-based signal 50a (and/or fluid from an additional fluid source), for instance air or a liquid, towards the actuator 40 to act as the opposition force 50b, thus allowing the user interface 41 to regain its original form after the patient has completed an activation action. Other opposition forces 50b may be contemplated as well.

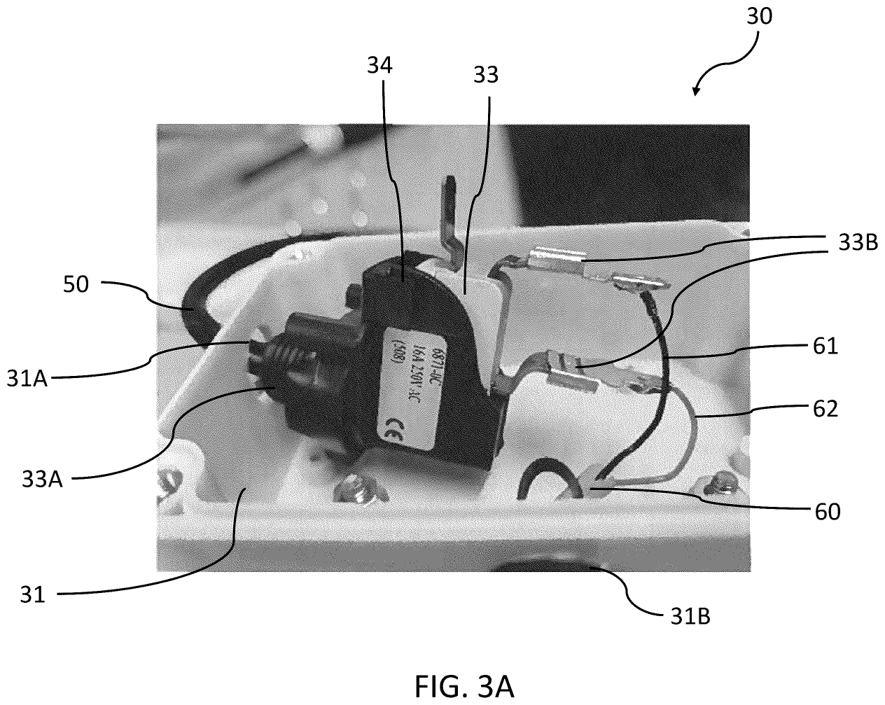

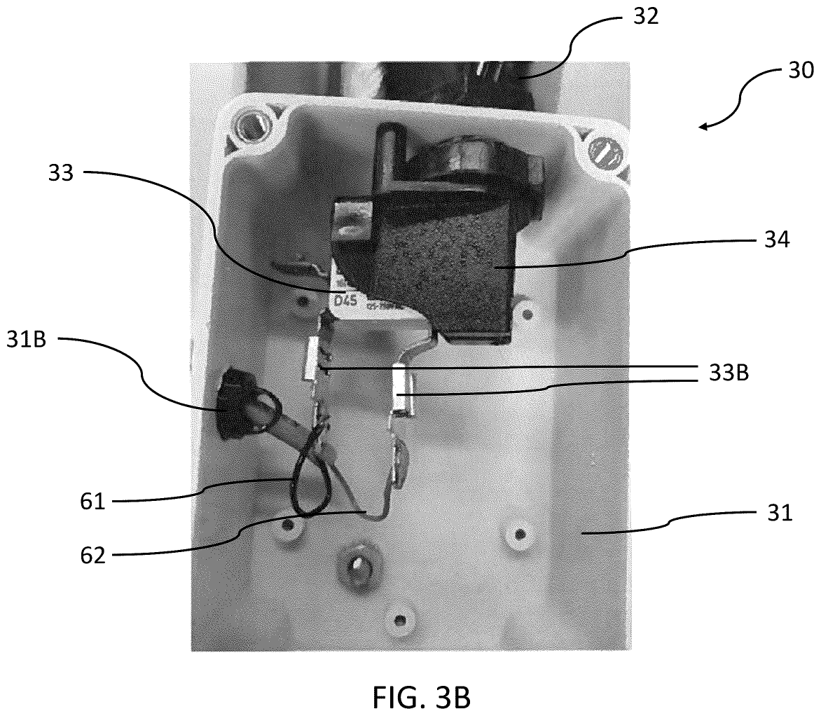

Referring additionally to FIGS. 3A-3B, an exemplary embodiment of the converter 30 is shown. As discussed above, the converter 30 includes the enclosure 31, illustratively shown in an open configuration (i.e., with an access door or panel removed), exposing an inner cavity or chamber of the enclosure 31. A signal converter 33 is disposed within the enclosure 31 and is configured for converting the fluid-based signal 50a received from the actuator 40 via fluid line 50 to an electrical-based signal to be transmitted to the infusion pump 20 via electrical line 60. In the shown embodiment, the signal converter 33 is configured for receiving a pneumatic signal from the pneumatic tubing 50 and converting the pneumatic (or air) signal to an electrical signal 60a. The signal converter 33 may thus be referred to as an air switch. Other signal converters may be contemplated, for instance to convert a hydraulic signal to an electrical signal. Stated differently, the signal converter 33 converts a mechanical force to an electrical signal.

Signal converter 33 illustratively includes an air inlet 33A and electrical terminals 33B, and is disposed in a casing or holder 34 which may operatively couple and retain the signal converter 33 to the enclosure 31. Alternatively, signal converter 33 may be directly mountable inside the enclosure 31 without the need for casing 34. Air inlet 33A is fluidly coupled to a second end of the pneumatic tubing 50, which enters the enclosure 31 via inlet 31A, though the air inlet 33A may be on the exterior of the enclosure 31 as another possibility. As discussed above, signal converter 33 may include means for redirecting the fluid-based signal 50a (or directing fluid from an additional fluid source) towards the actuator 40 as an opposition force 50b after the fluid-based signal 50a is received and registered by the signal converter 33. Such means may include a bladder or diaphragm (not shown) in an inside chamber of the signal converter 33. Electrical terminals 33B are electrically connected to positive and negative wires 61, 62 forming electrical line 60, which exits the enclosure 31 via outlet 31B. A third electrical terminal 33B may be provided, for instance to act as a ground or to transmit information to another device. It is thus understood that signal converter 33 may receive a fluid-type signal or like mechanical force such as a cable pull, for instance a pneumatic pulse, at inlet 33A, convert the fluid-type signal 50a to an electrical signal 60a, and close the appropriate circuitry for the electrical signal 60a to pass through electrical terminals 33B to positive and negative wires 61, 62 forming electrical line 60. Hence, in such embodiment, the signal converter 33 is passive. In another embodiment, the signal converter 33 could include a battery and emit a signal.

The converter 30 may further include notification or alerting means 35 to alert the patient when a signal from the actuator 40 has been successfully received at the converter 30. For instance, additional circuitry (not shown) may be operatively connected to the signal converter 33 and be configured to engage the connected alerting means 35 when fluid from the fluid line 50 is received at the signal converter 33. In an embodiment, said alerting means 35 (schematically shown as being mounted to the outside of enclosure 31) may be a light-emitting device to signal to the patient that the emitted signal from the actuator 40 has been received. Other locations for the light-emitting device may be contemplated, for instance to optimize visibility for the patient. The circuitry may additionally include a capacitor, a time-delayed relay, etc to allow the light-emitting device to remain illuminated for an extended period of time after the user interface 41 has been released to ensure that the patient is given sufficient time to notice the alert. Additionally or alternatively, the alerting means 35 may include a speaker or other sound emitting device operatively connected to the additional circuitry to audibly alert the patient that their signal has been received. This sound emitting device may, for instance, be disposed inside the enclosure 31. Other notification or alerting means, for instance a vibrating device operatively connected to the patient recovery bed, may be contemplated.

While FIG. 1 shows the converter 30 being a distinct component from the infusion pump 20 and actuator 40 and connected to the pump 20 via electrical line 60 and the actuator 40 via fluid line 50, other variations may be contemplated. For instance, the converter 30 may be mounted directly to the infusion pump 20. In such a case, the signal converter 33 may be operatively connected directly to the pump 20 (i.e., the electrical terminals 33B transmitting directly to the pump 20) or via a jack, or connector configured for the infusion pump 20, and thus the fluid-based signal from the actuator 40 may be converted and received at the infusion pump 20, with the electrical line 60 omitted. Alternatively, the converter 30 may be mounted directly to the actuator 40. For instance, the user interface 41 may be fluidly connected directly to the air inlet 33A of the signal converter 33, and thus actuation of the actuator 40 and conversion of the fluid-based signal to the electrical signal may occur at the actuator 40, with the fluid line 50 omitted. Other variations may be contemplated as well.

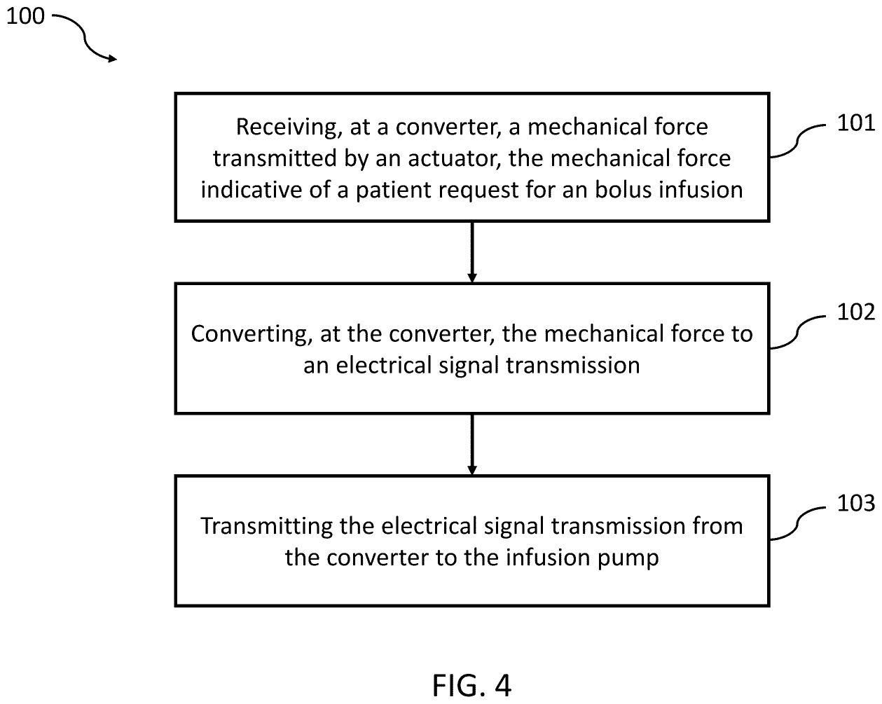

Referring to FIG. 4, there is shown a flowchart of an exemplary method 100 for self-administering a dose through the operation of an infusion pump, for instance infusion pump 20. At step 101, a mechanical force transmitted by an actuator 40 is received, at a converter 30, the mechanical force indicative of a patient request for a bolus infusion. In some embodiments, receiving the mechanical force may include receiving a fluid-based signal 50a such as a pneumatic signal from the actuator 40. The fluid-based signal 50a may be hydraulic as a possibility. At step 102, the mechanical force is converted, at the converter 30, to an electrical signal 60a transmission or emission. In some embodiments, the converter 30 includes an air switch configured for converting a pneumatic signal to an electrical signal 60a or electrical signal transmission. At step 103, the electrical signal 60a is transmitted from the converter 30 to the infusion pump 20. Various additions or modifications to method 100 may be contemplated.

It can be appreciated from the foregoing that at least some embodiments of the present disclosure include a control system for an infusion pump including an ergonomic actuator for selectively transmitting a fluid-based signal indicative of a patient's request for a bolus infusion to a pneumatic-electronic converter which transmits an electronic signal to the infusion pump, thereby allowing a patient with limited mobility to self-regulate the dosage of their medication. The patient may control the delivery of medication, for instance pain medication, based on their own judgement of their needs, for instance their current level of pain. The dosage of such medication may thus be better synchronized with the needs of the patient.

As can be seen therefore, the examples described above and illustrated are intended to be exemplary only. The scope is indicated by the appended claims.

What is claimed is:

1. A control system for an infusion pump, comprising:
a converter coupled to the infusion pump; and
an actuator configured for receiving a mechanical force, and for selectively transmitting the mechanical force to the converter indicative of a patient's request for a bolus infusion;
wherein the converter is configured for converting the mechanical force received from the actuator to an electrical signal transmission to the infusion pump.

2. The control system as defined in claim 1, wherein the actuator is configured for selectively transmitting a fluid-based signal to the converter.

3. The control system as defined in claim 2, wherein the fluid-based signal is a pneumatic signal.

4. The control system as defined in claim 3, wherein the converter includes an air switch configured for converting the pneumatic signal to the electrical signal.

5. The control system as defined in claim 2, wherein the actuator includes a bladder fluidly connected to the converter via a fluid line.

6. The control system as defined in claim 1, wherein the converter is disposed in an enclosure mounted to a solute pole for the infusion pump.

7. The control system as defined in claim 6, wherein the enclosure is substantially waterproof.

8. The control system as defined in claim 1, wherein the actuator is mounted to a patient recovery bed.

9. The control system as defined in claim 8, wherein the actuator is mounted to a guard rail of the patient recovery bed.

10. The control system as defined in claim 1, wherein the actuator has a width of at least 9 centimeters.

11. The control system as defined in claim 1, wherein the converter is further configured for transmitting an opposing mechanical force to the actuator subsequently to receiving the mechanical force.

12. A method for controlling the operation of an infusion pump, comprising:
receiving, at a converter, a mechanical force transmitted by an actuator, the mechanical force indicative of a patient request for an bolus infusion;
converting, at the converter, the mechanical force to an electrical signal transmission; and
transmitting the electrical signal from the converter to the infusion pump.

13. The method as defined in claim 12, wherein receiving the mechanical force transmitted by the actuator includes receiving a fluid-based signal transmitted by the actuator.

14. The method as defined in claim 13, wherein receiving the fluid-based signal transmitted by the actuator includes receiving a pneumatic signal transmitted by the actuator.

15. The method as defined in claim 14, wherein the converter includes an air switch, and converting the mechanical force to the electrical signal transmission further includes converting the pneumatic signal to the electrical signal transmission.

16. The method as defined in claim 13, wherein the actuator includes a bladder fluidly connected to the converter via a fluid line, whereby receiving the mechanical force includes receiving a fluid pressure from the bladder.

17. The method as defined in claim 12, further comprising positioning the converter in a waterproof enclosure mounted to a solute pole for the infusion pump.

18. The method as defined in claim 12, further comprising mounting the actuator to a patient recovery bed.

19. The method as defined in claim 12, further comprising transmitting, by the converter, an opposing mechanical force to the actuator subsequently to the receiving the mechanical force transmitted by the actuator.
